(19) United States
(12) Patent Application Publication
(10) Pub. No.: US 2006/0293732 A1
(43) Pub. Date: Dec. 28, 2006

(54) Thermoelectric cooler (TEC) heat exchanger for intravascular heat exchange catheter

(76) Inventors: Kenneth A. Collins, Mission Viejo, CA (US); Grant Palmer, Irvine, CA (US)

Correspondence Address: ROGITZ & ASSOCIATES, 750 B STREET, SUITE 3120, SAN DIEGO, CA, 92101, US

(21) Appl. No.: 11/168,087

(22) Filed: Jun. 27, 2005

(51) Int. Cl. A61F 7/00

(52) U.S. Cl. 607/104

(57) ABSTRACT

A patient temperature control catheter or pad is warmed or cooled by coolant circulating through it. The coolant in turn is warmed or cooled as it flows through a cartridge that is placed against a control system TEC. For cooling, heat is removed from the TEC by tap water flowing through a reservoir that is in contact with the TEC. If desired, a small turbine can be disposed in the reservoir to be driven by the flowing tap water, and the turbine is coupled to a pump element that pumps the coolant through the catheter or pad.

### I. FIELD OF THE INVENTION

The present invention relates generally to patient temperature control systems.

### II. BACKGROUND OF THE INVENTION

It has been discovered that the medical outcome for a patient suffering from severe brain trauma or from ischemia caused by stroke or heart attack or cardiac arrest is improved if the patient is cooled below normal body temperature (37° C.). Furthermore, it is also accepted that for such patients, it is important to prevent hyperthermia (fever) even if it is decided not to induce hypothermia. Moreover, in certain applications such as post-CABG surgery, it might be desirable to rewarm a hypothermic patient.

As recognized by the present invention, the above-mentioned advantages in regulating temperature can be realized by cooling or heating the patient's entire body. Moreover, the present invention understands that since many patients already are intubated with central venous catheters for other clinically approved purposes anyway such as drug delivery and blood monitoring, providing a central venous catheter that can also cool or heat the blood requires no additional surgical procedures for those patients. The following U.S. patents, all of which are incorporated herein by reference, disclose various intravascular catheters/systems/methods: U.S. Pat. Nos. 6,749,625, 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559, and U.S. patent application Ser. No. 10/355,776. Less optimally, surface cooling can be used. U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein by reference, disclose such surface cooling systems. In both intravascular catheters and external pad systems, coolant such as a gas or saline is circulated through the heat exchange element.

Regardless of the particular heat exchange element that is engaged with the patient, it is clear that heat must be removed from or added to the coolant that flows through the heat exchange element. The present invention makes the following critical observations. Hospital space is at a premium; thus, compact systems are desired. Nonetheless, it is desirable that while small size is to be sought in a system for heating or cooling the coolant that flows through the catheter or external pad, it is also desired that the system have a high heat removal capacity, because it is often desirable to cool a patient as rapidly as possible. With these critical observations in mind, the invention herein is provided.

### SUMMARY OF THE INVENTION

A system for exchanging heat with a patient temperature control element such as an intravascular closed loop catheter or an externally applied pad includes a secondary heat exchange element such as but not limited to cartridge, and primary coolant such as but not limited to saline flows in a closed loop which includes the secondary heat exchange element and patient temperature control element. A thermoelectric cooler (TEC) assembly is in thermal contact with the secondary heat exchange element. A reservoir receives tap water from a building tap water system and is in thermal contact with the TEC assembly to remove heat from the TEC assembly.

In some implementations a turbine can be provided in the reservoir to be driven by flowing tap water. The turbine is coupled to a pump element that pumps the coolant through the closed loop. Tap water intake and exhaust lines can communicate with the reservoir and at least one valve can be disposed in one of the intake and exhaust lines. A controller may be provided for establishing a power level to the TEC assembly based on a desired patient temperature and an actual patient temperature. The controller can also control the tap water valve.

In another aspect, a method for altering patient temperature includes engaging an intravascular catheter and/or an external heat exchange pad with a patient, and causing coolant to flow through the catheter and/or pad without the coolant touching the patient. The coolant exchanges heat with the patient. The method also includes exchanging heat with the coolant using a TEC assembly and selectively removing heat from the TEC assembly using tap water in a building.

In still another aspect, a heat exchange system has primary means engageable with the body of a patient to exchange heat therewith, primary heat transfer means, a TEC assembly, and secondary means juxtaposable with the TEC assembly to exchange heat therewith. The primary heat transfer means flows in a closed loop between the primary means and secondary means to transfer heat therebetween. Liquid means remove heat from the TEC assembly.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic diagram showing two heat exchange modalities; and

FIG. 2 is a block diagram of a non-limiting implementation of the control system.

### DETAILED DESCRIPTION OF THE PREFERRED EMBODIMENT

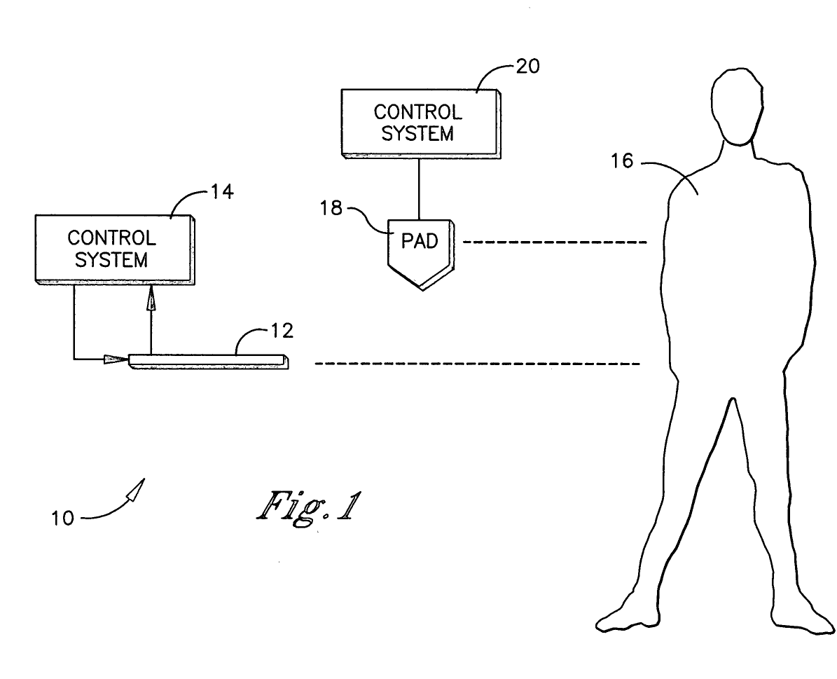

Referring initially to FIG. 1, a system is shown, generally designated 10, that may include a heat exchange catheter 12 that is in fluid communication with a catheter temperature control system 14.

In accordance with present principles, the system 10 can be used to induce therapeutic hypothermia in a patient 16 using a catheter in which coolant such as but not limited to saline circulates in a closed loop, such that no coolant enters the body. While certain preferred catheters are disclosed below, it is to be understood that other catheters can be used in accordance with present principles, including, without limitation, any of the catheters disclosed in the following U.S. patents, all incorporated herein by reference: U.S. Pat. Nos. 5,486,208, 5,837,003, 6,110,168, 6,149,673, 6,149,676, 6,231,594, 6,264,679, 6,306,161, 6,235,048, 6,238,428, 6,245,095, 6,251,129, 6,251,130, 6,254,626, 6,261,312, 6,312,452, 6,325,818, 6,409,747, 6,368,304, 6,338,727, 6,299,599, 6,287,326, 6,126,684. The catheter 12 may be placed in the venous system, e.g., in the superior or inferior vena cava.

Instead of or in addition to the catheter 12, the system 10 may include one or more pads 18 that are positioned against the external skin of the patient 16 (only one pad 18 shown for clarity). The pad 18 may be, without limitation, any one of the pads disclosed in the external pad patents. The temperature of the pad 18 can be controlled by a pad controller 20 in accordance with principles set forth in the external pad patents to exchange heat with the patient 16, including to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia. The control systems 14, 20 may be implemented by a single system.

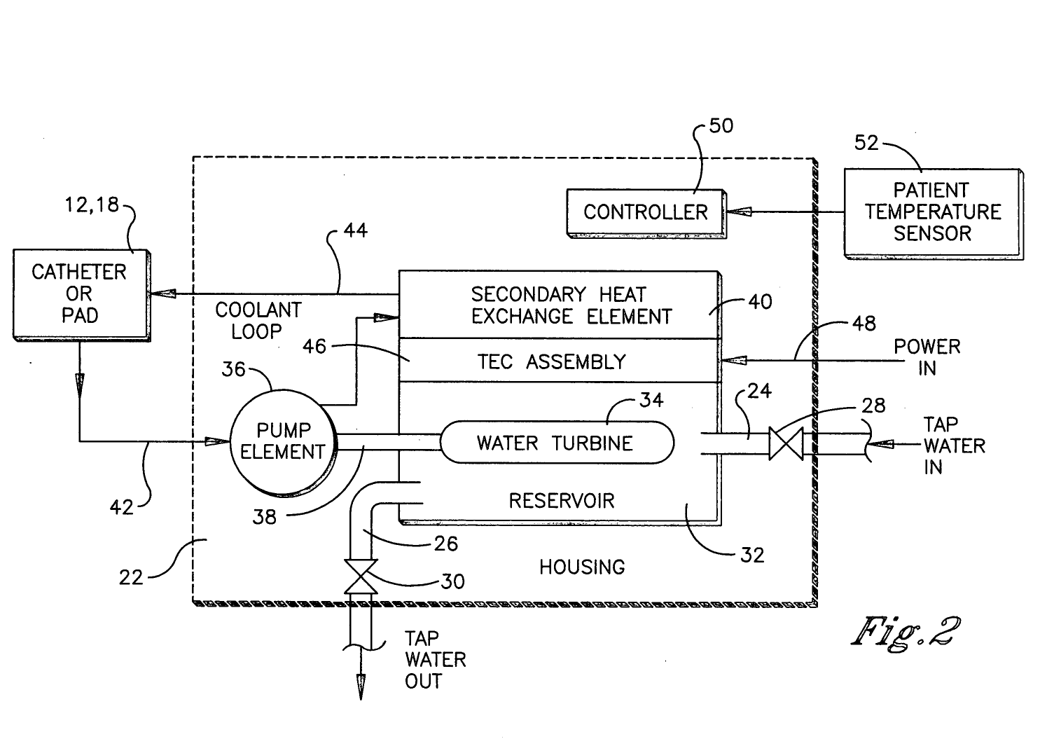

Now referring to FIG. 2, a control system of the present invention may be seen. A primary heat exchange element such as the catheter or pad 12, 18 is connected to a system housing 22. The housing 22 includes a tap water intake line 24 and a tap water exhaust line 26 that can be connected to the tap water system and drainage system, respectively, of a hospital or other medical establishment in which the housing 22 is located. By “tap water” system is meant any water system in the hospital that is piped through the structure, including tap water and, if provided, a purified water system.

Tap water intake and exhaust isolation valves 28, 30 may be respectively in intake and exhaust lines 24, 26 as shown. One or both valves 28, 30 may be controlled by the controller described below and, hence, may be, without limitation, solenoid-operated valves.

FIG. 2 shows that the tap water intake and exhaust lines 24, 26 communicate with a tap water reservoir 32. In some implementations a small water turbine 34 may be disposed in the reservoir 32 such that when tap water flows from the intake line 24 to the exhaust line 26, the water turns the turbine 34. The turbine 34 is coupled to a pump element 36 as shown through a shaft 38 or other coupling mechanism. The pump element may be, without limitation, an impeller, a cam-based peristaltic “finger” pump, a gear-type pump, or other pumping device. In any case, the pump element 36 provides the motive force for the primary coolant circuit. Specifically, the pump element 36 circulates coolant between the catheter or pad 12, 18 and a secondary heat exchange element 40 which may be, without limitation, a cartridge-type body that has tubes or channels through which the coolant flows and which can be made of high thermal efficiency thermoplastic or metal. In the non-limiting embodiment shown the pump element 36 is disposed in a coolant return line 42 from the catheter or pad, and it pumps coolant through the secondary heat exchange element 40 and out of a coolant supply line 44 back to the catheter or pad in a closed loop. Alternatively, the pump element 36 may be provided in the coolant supply line 44.

To remove heat from or add heat to the secondary heat exchange element 40, a thermoelectric cooler (TEC) assembly 46 is provided between the secondary heat exchange element 40 and the tap water reservoir 32 in dry thermal contact with both. In non-limiting embodiments the reservoir 32 is in physical contact with the TEC assembly 46. The TEC assembly 46 functions to heat or cool in accordance with principles known in the art when it receives appropriate electrical power 48 under control of a controller 50. Without limitation, the TEC assembly 46 may any of the assemblies disclosed in the following U.S. patents, incorporated herein by reference: U.S. Pat. Nos. 6,019,783, 6,436,130, 6,149,676, 6,635,076. The controller 50, which may be implemented by a digital microprocessor, can execute, by way of non-limiting example, any of the patient temperature control algorithms in the above-referenced patents and may be implemented by any of the controllers in any of the patents referenced herein. To that end, the controller 50 may receive a temperature signal from any suitable patient temperature sensor 52 as feedback.

With the above structure in mind, it may now be appreciated that when it is desired to cool the patient, the controller 50 opens the valves 28, 30, which not only cools the TEC assembly 46 but also turns the turbine 34 and, hence, actuates the pump element 36. The controller 50 also controls power to the TEC to cause it to become cold on the surface facing the secondary heat exchange element 40, with the magnitude of the power to the TEC controlled to vary with the magnitude of the difference between desired and actual patient temperature. Coolant thus flows through the catheter or pad 12, 18 under the influence of the pump element 36 and exchanges heat with the TEC assembly 46 as it flows through the secondary heat exchange element 40. In turn, heat is removed from the TEC assembly 46 by the tap water flowing through the reservoir 32. To warm the patient, current flow through the TEC assembly is simply reversed. Because liquid (tap water) is used to cool the TEC, the system 10 does not require relatively bulky and noisy air cooling apparatus.

The secondary heat exchange element 40 may be supplied with the catheter or pad and may be disposable. In this case, a caregiver would engage the secondary heat exchange element 40 with the control system 20 using suitable connections such as Luer fittings for the fluid lines and clips, etc. to hold the secondary heat exchange element 40 in place against the TEC assembly 46. The primary coolant supply and return line, as appropriate, can be tubing lines that are placed in the pumping chamber of the pump element 36 when, for instance, the pump element 36 is a finger-type peristaltic pump. Or, the pump element 36 can be included in the primary coolant assembly and engaged with the coupling 38 upon association of the primary coolant circuit with the control system 20. The control system 20 may be mounted on the wall of a hospital critical care room, or within the wall, so that control circuitry associated with the controller 50 such as a touch sensitive flat panel screen would be the only part of the control system 50 to appear on the wall.

While the particular THERMOELECTRIC COOLER (TEC) HEAT EXCHANGER FOR INTRAVASCULAR HEAT EXCHANGE CATHETER as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. It is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. Absent express definitions herein, claim terms are to be given all ordinary and accustomed meanings that are not irreconcilable with the present specification and file history.

What is claimed is:

1. A system for exchanging heat with a patient temperature control element, comprising:
at least one secondary heat exchange element, coolant flowing in a closed loop including the secondary heat exchange element and patient temperature control element;
at least one thermoelectric cooler (TEC) assembly in thermal contact with the secondary heat exchange element; and
at least one reservoir receiving tap water from a building tap water system and in thermal contact with the TEC assembly.

2. The system of claim 1, comprising a turbine in the reservoir and actuated by flowing tap water, the turbine being coupled to a pump element that pumps the coolant through the closed loop.

3. The system of claim 1, wherein the patient temperature control element is an intravascular catheter.

4. The system of claim 1, wherein the patient temperature control element is a pad configured for external application against the patient's skin.

5. The system of claim 1, comprising tap water intake and exhaust lines communicating with the reservoir and at least one valve disposed in one of the intake and exhaust lines.

6. The system of claim 1, comprising a controller establishing a power level to the TEC assembly at least in part based on a desired patient temperature and an actual patient temperature.

7. The system of claim 5, comprising a controller controlling the valve(s).

8. A method for altering patient temperature, comprising:
engaging an intravascular catheter and/or an external heat exchange pad with a patient;
causing coolant to flow through the catheter and/or pad without the coolant touching the patient, the coolant exchanging heat with the patient;
exchanging heat with the coolant using a TEC assembly; and
selectively exchanging heat with the TEC assembly using tap water in a building.

9. The method of claim 8, wherein the causing act is undertaken at least in part by directing tap water through a reservoir in thermal contact with the TEC assembly.

10. The method of claim 8, comprising opening and closing at least one valve as desired to allow tap water to flow into a reservoir in thermal contact with the TEC assembly.

11. The method of claim 8, comprising establishing a power level to the TEC assembly at least in part based on a desired patient temperature and an actual patient temperature.

12. A heat exchange system, comprising:
primary means engageable with the body of a patient to exchange heat therewith;
primary heat transfer means;
a thermoelectric cooling (TEC) assembly;
secondary means juxtaposable with the TEC assembly to exchange heat therewith, the primary heat transfer means flowing in a closed loop between the primary means and secondary means to transfer heat therebetween; and
liquid means for removing heat from the TEC assembly.

13. The system of claim 12, wherein the primary means is an intravascular closed loop catheter.

14. The system of claim 12, wherein the primary means is an externally applied heat exchange pad.

15. The system of claim 12, wherein the primary heat transfer means is saline.

16. The system of claim 12, wherein the liquid means is tap water.

17. The system of claim 16, wherein the tap water flows through a reservoir that is in thermal contact with the TEC assembly.

18. The system of claim 17, comprising means for causing the primary heat transfer means to flow, the means for causing being disposed in the reservoir and being actuated by flowing tap water in the reservoir.
